(19) United States
(12) Patent Application Publication
(10) Pub. No.: US 2009/0221057 A1
(43) Pub. Date: Sep. 3, 2009

(54) Bio-Breeder System for Biomass Production

(76) Inventor: James C. Kennedy, St. Louis, MO (US)

Correspondence Address: GLENN L. WEBB;GLENN L. WEBB P.C., P.O BOX 3788, DURANGO, CO, 81302, US

(21) Appl. No.: 12/039,444

(22) Filed: Feb. 28, 2008

(51) Int. Cl. C12M 1/36

(52) U.S. Cl. 435/286.5

(57) ABSTRACT

A system for converting flue gases into useful biofuels and oxygen. The system uses flue gases and thermal energy from smokestack industries to grow algae in a controlled environment. The flue gases are used as nutrients for the growth of algae through photosynthesis which is then processed into biofuels. The algae growth occurs in a controlled environment to minimize contamination of the algae as well as to optimize the algae growth. Sunlight and artificial light is modulated to provide the optimum light source for the photosynthesis process.

### FIELD OF THE INVENTION

This invention relates to the field of biological breeders for algae production and for reducing greenhouse gases.

### BACKGROUND OF THE INVENTION

Two major related issues are of major concern at the present time. One of these issues is the production of greenhouse gases and their effect on the environment. The second issue is the use of fossil fuels and their effect on the environment and economy. These issues and the ability to deal with these issues are of paramount concern not only in the United States but also in worldwide.

Greenhouse gases are components of the atmosphere that contribute to the the greenhouse effect, that is, the absorption of the longer wavelengths of sunlight in order to warm the earth. The greenhouse gases include water vapor, carbon dioxide, methane, nitrous oxide, sulfur hexaflouride, hydrofluorocarbons, perfluorocarbons and chlorofluorocarbons. The increase of greenhouse gases into the atmosphere, according to many scientific studies, has led to global warming leading to climate change. A major source of anthropogenic greenhouse gases is the burning of fossil fuels, primarily from coal and gasoline. The burning of fossil fuels in particular have lead to great increases of carbon dioxide released into the atmosphere along with the release of sulfur hexafluoride and nitrous oxide. These released gases may remain in the atmosphere for over 100 years accumulating through that entire time.

Carbon dioxide is naturally converted into biomass through photosynthesis. However, the reduction of forests and grasslands along with the increase in the production of greenhouse gases including carbon dioxide has led to the accumulation of greenhouse gases in the atmosphere. There have been a number of attempts to reduce this accumulation of greenhouse gases through such methods as sequestering these gases underwater or in caverns. This has largely been unsuccessful due to the expense and temporal nature of such attempts. Also the sequestered gases are unavailable for conversion into energy sources.

Recently, there has been interesting research into bio-regeneration of greenhouse gases into biomass through algae photosynthesis. One research study (Sheehan, John, Dunahay, Terri, Benemann, John R., Roessler, Paul, “A Look Back at the U.S. Department of Energy's Aquatic Species Program: Biodiesel from Algae,” 1998,) found that over ninety percent of carbon dioxide fed to algae can be absorbed. The algae can then be processed into biofuels, such as methane through gasification processes, ethanol through fermentation, biodiesel as well as other fuels.

Most of the research associated with bio-regeneration of greenhouse gases into biomass have utilize large open water ponds. The flue gases are pumped into these ponds to allow the algae to capture the gases and convert them into biomass. However, the uncontrolled environment of these open water systems leads to low algae production as well as difficulties in harvesting of the algae.

One attempt to commercialize the bio-regeneration of greenhouse gases through algae is disclosed in U.S. Pat. No. 6,667,171, Bayless et al. Bayless et al. uses a membrane contained in a closed tank CO₂ and NOx is piped into the tank as a feedstock for algae grown on the membrane. The algae is then harvested by cleaning the membrane.

Another attempt is disclosed in U.S. Patent Application Publication No. US 2005/0260553, Berzin. This application discloses a photobioreactor that uses translucent conduits with flue gases such as CO₂ and NOx circulated within a liquid medium to grow algae. This system also uses photomodulation to achieve high growth cycles of algae.

A need exists to improve the efficiency and utility of bio-regeneration systems to reduce the amount of flue gases entering the atmosphere, to utilize thermal energy that is being wasted and for increasing the amount of biofuels recovered from the flue gases.

### SUMMARY OF THE INVENTION

The present invention provides a bio-regeneration system that uses photosynthesis to recover carbon from flue gases in a form that can be utilized to create biofuels. The present invention is also able to reduce the amount of flue gases entering the atmosphere. The present invention provides a biological breeder for growing algae. The biological breeder uses flue gases captured from industrial sites as nutrients to grow the algae. This prevents those gases from entering the atmosphere. Thermal energy from those gases may also be used to power the biological breeder and to process the algae into biofuels.

The system of a preferred embodiment of the present invention utilizes a controlled environment to prevent contamination of the algae as well as to increase the growth rate of the algae. The controlled environment can be a mine, a cave, an indoor facility or any other facility that allows the environment around the algae breeder to be controlled as to the optimum temperature for algae growth as well as to prevent contamination of the algae.

The algae breeder system of a preferred embodiment utilizes light from solar collectors as well as artificial light sources directed into the algae breeder to facilitate the photosynthesis process. The light sources are modulated to create the optimum light frequency, color spectrum, strobe effect, light cycle and other factors to optimize the growth of the algae in the breeder.

A series of light rings and strands are used to evenly distribute the light throughout the liquid medium in the tank to optimize the growth of the algae. A cleaning mechanism is used to periodically clean the light strands from algae growth.

The inner surfaces of the tank, in a preferred embodiment, form a reflective surface to ensure that the light is evenly distributed through out the tank. A scraper mechanism is used to periodically clean the inner surfaces to prevent algae buildup causing uneven light distribution.

The algae breeder system of a preferred embodiment uses flue gases containing carbon dioxide as nutrients for growing the algae in the breeder tank. The flue gases are sparged to create small bubbles that rise upward through the tank to provide nutrients for the algae in a liquid medium in the tank.

The algae is harvested and processed into biofuels. The optimized growth of the algae greatly increases the yield of carbon in the algae which also means that less pollution from the flue gases has occurred.

These and other features of the present invention will be evident from the ensuing detailed description of preferred embodiments, from the claims and from the drawings.

### BRIEF DESCRIPTION OF THE DRAWINGS

FIG. 1 is a diagram of the process occurring to convert the flue gases into oxygen and biomass.

FIG. 2 is a schematic of the algae breeder system of a preferred embodiment of the present invention.

FIG. 3 is a partial view of the light system of the algae breeder of the embodiment of FIG. 2.

FIG. 4 is a top view of the light system of FIG. 3.

FIG. 5 is a cutaway view of the tank of the embodiment of FIG. 2.

FIG. 6 is another cutaway view of the tank of FIG. 5.

### DETAILED DESCRIPTION OF PREFERRED EMBODIMENTS

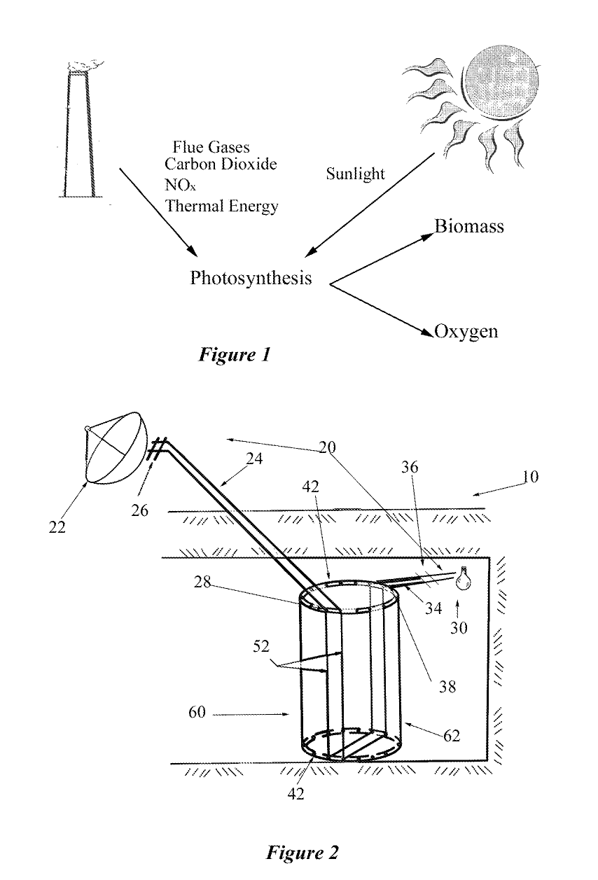

The present invention provides a unique application for sequestering greenhouse gases from polluting the atmosphere and for producing alternative energy sources. The present invention does by providing a biological breeder that utilizes the sequestered gases and excess thermal energy from a utility or industrial application for growing algae that can be harvested into an alternative energy source. A preferred embodiment of this invention is illustrated in FIGS. 1-6. It is to be expressly understood that the descriptive embodiments are provided herein for explanatory purposes only and are not meant to unduly limit the claimed inventions. The exemplary embodiments describe the present invention in terms of a biological co-generation system for growing algae with flue gases. The system may also be used with any type of algae systems.

### Overview

The biological algae breeder (bio-breeder) of a preferred embodiment of the present invention is utilized in a co-generation manner with an industrial application that is producing excess heat and flue gases, including without limitation carbon dioxide, nitrogen oxides, sulfur oxides and other gases that may contribute to polluting and/or the atmospheric “greenhouse” effect. These applications include without limitation coal-powered electrical generation utilities, iron ore smelting applications and many other applications. The bio-breeder utilizes excess thermal energy to control the environment of the bio-breeder as described below and utilizes the flue gases as feed stocks for the algae in the bio-breeder. Other sources of thermal energy and gases may be used as well.

The bio-breeder of the present invention provides a closed environment for controlling the growth of the algae for optimum results as shown in the workflow diagram in FIG. 1. Flue gases and other nutrients are introduced into a liquid medium containing algae feedstock. Light is provided into the liquid medium from solar and/or artificial light sources. Algae in the liquid medium is grown through photosynthesis to convert the carbon dioxide and other nutrients into oxygen and carbon biomass.

The overall environment is controlled to prevent contamination of the system as well as to optimize the growth cycle of the algae. Algae is typically optimally grown at about 27 degrees centigrade. Flue gases are provided as feedstock for the algae in the closed system to reduce the introduction of pollutants and greenhouse gases into the atmosphere and to convert the carbon from those gases through photosynthesis into a usable fuel source. Thermal energy from those flue gases is also used to heat the system if necessary as well as used for energy for processing the algae. Sunlight is collected and modulated to increase the growth cycle of the algae. Artificial light may also be combined to supplement and/or replace the sunlight. The sunlight and artificial light are modulated to provide the optimum spectrum, frequency and other factors for the growth of the algae.

The biological breeder of a preferred embodiment of the present invention is illustrated in FIG. 1. The breeder system 10 includes lighting system 20, breeder reactor 60 and flue feed gas feed 80. Algae is grown within the reactor 60 under constant light from lighting system 20 and provided nutrients in the form of waste gases captured from smokestacks such as coal powered electrical plants. In the preferred embodiment, the reactor 50 is contained in a controlled environment, such as underground or indoors, in order to provide a constant and stable environment. Thermal waste heat is also provided from the smokestack for temperature control within the environment as well as for fermentation, distillation and drying of the pulp harvested from the algae grown in the reactor.

### Lighting System

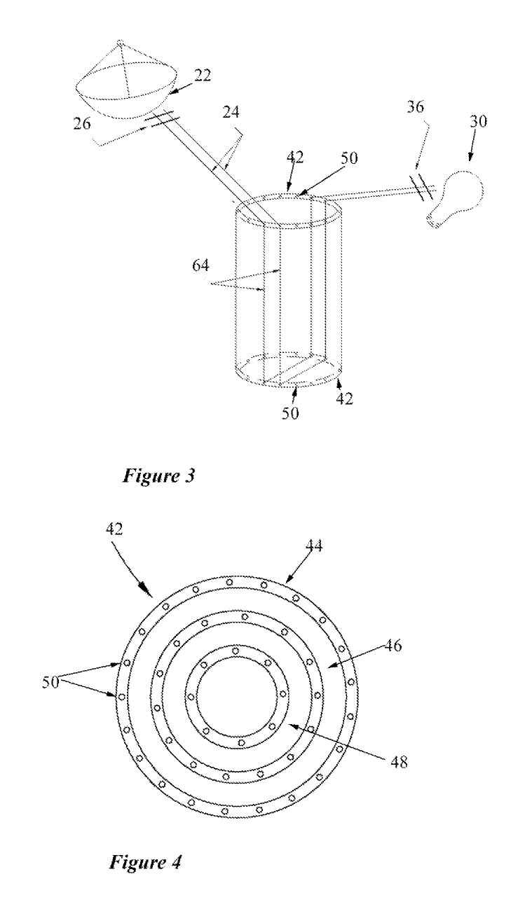

Lighting system 20, in the preferred embodiment as shown in FIGS. 2 and 3, includes self tracking solar collectors 22. The self tracking solar collectors are able to track the relative movement of the earth and sun to optimize the maximum amount of sunlight directed into the collectors. Each of the self tracking solar collectors focus incoming sunlight into delivery channels 24 which may be fiber optics or any other suitable light transmitting channel. The delivery channels are connected to one or more of a series of nodes 26. These nodes 26 may include lenses, filters, shutters or other light manipulation devices. The nodes 26 include one-way mirrors, a polarizing filter to filter out harmful frequencies that may cause the algae in the reactor 60 to become photo-inhibited. The nodes 26, either individually or collectively, also provide shuttering capabilities to the light, so that the transmitted light can pass in alternate light and dark phases into the reactor 60. This can create a strobe effect for the transmitted light. These nodes 26 may also alter the color of the light as well as alter the light spectrum in order to optimize the light for the algae being grown within reactor 60. The light modulation effect from the nodes 26 are able to multiply the growth cycles of the algae to increase their growth rate.

Each of the delivery channels 24 also include one-way mirrors 28 to prevent light loss from the reactor 20 back up the delivery channels 24. The delivery channels 24 after passing through one-way mirrors 28 are connected to an internal distribution lighting system 40 (discussed in greater detail below) within reactor 60.

Auxiliary lighting system 30 includes artificial light sources 32, such as light emitting diodes, fluorescent lights, incandescent lights or any other artificial light source, is also connected through delivery channels 34, such as fiber optics to the internal distribution lighting system 40. The delivery channels 34 pass through filter nodes 36 which filter and shutter the transmitted light from auxiliary light system 42. The delivery channels also pass through one way mirrors 38 before connecting to the internal distribution lighting system 40 to prevent light leakage from the reactor 60. In the preferred embodiment, the auxiliary lighting system connects to the opposing end of the internal distribution lighting system than the solar lighting system. The auxiliary light system 30 is intended to supplement the light from the solar light collectors 22 as well as to replace the transmitted sunlight during nightfall as well as overcast days.

### Reactor

The reactor or breeder 60 in a preferred embodiment is a tank 62 of any dimension or configuration and can be formed from any material, preferably opaque. The tank in the preferred embodiment is mounted in a vertical configuration but could be horizontal or angular as well. The reactor 60 has a reflective inner surface to prevent light loss from the lighting system 20 within the tank. Internal distribution lighting system 40 is mounted on the opposing ends of the tank 62.

Internal distribution lighting system 40 include crown rings 42 shown in FIGS. 2 and 3 mounted on each end of the tank 50. The crown rings are separated from one another by guide rods 64 that are spaced around the inner periphery of tank 62. The crown rings 42 include a series of concentric rings 44, 46, 48. The number of rings depend on the size and configuration of the tank 62, the amount of desired light, the properties of the algae being grown and other factors. One embodiment uses rings spaced radially six to twelve inches from one another in an eight foot diameter tank. For descriptive purposes only, the illustrated embodiment uses three concentric rings.

The concentric rings include securing mechanisms, such as eyelets 50, for attaching fiber optics and/or light emitting diode (LED) strands 52 between each of the opposing crown rings 42. These strands or cables 52 are etched or scribed to provide even passage of all available light into the reactor within the crown rings 42. The eyelets 50 include cleaning mechanisms 54, such as grommets, for cleaning algae from the strands 52. This cleaning action ensures that the light will be uniformly reflected throughout the tank 62.

Reactor 60 also includes crown scraper 66 shown in FIG. 4 that is mounted on the guide rods 64 to travel along the guide rods 64. The crown scraper can be powered by any actuation device such as a motor, cable, hydraulics, or any other type of actuation device or even manually. In the preferred embodiment, the outer surface 68 of the crown scraper 64 has a semi-rigid insert or other type of scraping device to scrape against the inner periphery of the tank 62. The movement of the crown scraper 66 along the guide rods 64 will scrape the algae from the inner periphery of the tank 62. This restores the reflectivity of the inner periphery of the tank 62. The grommets 54 on the strands 52 may be connected to the crown scraper 66 to clean the light strands 52 at the same time.

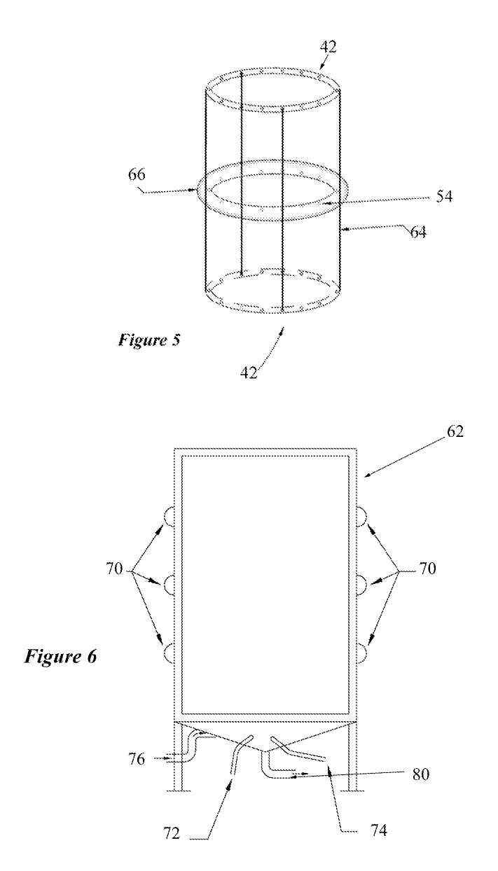

Sensors 70 are mounted on and in the tank 62 at appropriate locations as shown in FIGS. 2 and 5. These sensors measure the density of the algae within the tank 62, the differentiation of the density of the algae, the light levels within the tank, volumes within the tank and any other necessary parameters of the algae breeding operation. The sensors can transmit the sensed values to a controller or processor that can change the light modulation, light amplitude, increase or decrease the nutrients, water flow, scrape the tank and strands, increase or decrease the environmental temperatures, start the harvest process or any other process to optimize the algae growth.

Reactor 60 also includes nutrient delivery channel 72, carbon dioxide delivery channel 74, as well as additional delivery channels 76, 78 for delivering atmospheric air and other gasses from the smokestack, such as NOx, SOx and other flue gasses. The carbon dioxide and other gasses are delivered by micro-bubble spargers that will deliver the gases as bubbles smaller than five microns. The flow of gasses and nutrients from these channels into reactor 60 are controlled according to the measurements obtained from sensors 70.

The reactor 60 also includes an outlet discharge 80. Once the optimum density of the algae has been determined by the sensors 70, a portion of the content of the tank 62 will be drained away for processing. Typically, less than half of the total content of the reactor will be drained during the harvest. This will be optimized based on the growth rate of the algae, the type of algae and other factors.

The algae remaining after the harvesting process will then feed on new nutrients fed into the tank 62 with the replacement water and sparged gases. This process of refilling the reactor with nutrients, water and gases may be intermittent to ensure even suspension throughout the tank.

The entire operation of optimizing the lighting, the nutrients and the harvesting of the algae within the reactor 60 can be entirely automated for operation twenty four hours a days without interruption. A computer, processor or other type of controller can be used to receive values from the sensors 70 to control this operation.

The reactor 60, in the preferred embodiment, is mounted within a controlled environment. This controlled environment may be, as in the preferred embodiment, underground, such as in a mine, cave or excavated area, or indoors in a building. For example, closed mines in Pennsylvania or other mining states could be revivaltized to incorporate the breeders. These could be near coal powered utilities, iron ore smelters or other industries producing the feedstock gases. This allows not only the temperature of the reactor to be tightly controlled to optimize the growth of the algae, but also minimizes contamination of the algae by unwanted contaminates that might inhibit the growth of the algae. In particular, aquatic plant species or other organisms that might inhibit the algae growth that are often found in open algae ponds can be minimized in the closed system of the present invention. Thermal heat from the companion process providing the flue gases to the reactor may also be used to maintain the temperature around the reactor 60 for optimal growth of the algae.

The harvested lipoidal algae will be discharged to a processing station. The lipoidal algae effluent will be dewatered to form the algae into a pulp. The pulp can then be prepped for further processing, such as to create a biodiesel fuel, or dried for an alternative use. The thermal heat from the companion process that is supplying the flue gases can be used to power this process, to supply heat for the drying process and for drying the pulp.

### Operation

In operation, flue gases are captured from an industrial application and delivered to the chamber containing the breeder tank 60. Preferably the chamber is an underground cavern or mine but can be any installation where the environment can be controlled. The controlled environment minimizes contamination of the algae as well as providing the optimum environment for the growth of the algae. The flue gases are sparged by a micro-sparger (not shown) that creates bubbles, preferably less than five microns. The sparged gases are delivered into the tank 60 through channels 76, 78. The sparged gases rise through the liquid medium in the tank to provide nutrients to the algae growing the liquid medium. Excess gases can be vented from the top of the tank if necessary.

Light is transmitted into the tank from the solar collectors and/or the artificial light sources. This light is modulated to provide the optimum spectrum, frequency, strobe and any other variable of the light to facilitate the growth of the algae. The light is transmitted through the light crown rings 42 along the strands 52 to evenly transmit the light throughout the liquid medium. The inner surfaces of the tank reflect the light as well to ensure even transmission of the light throughout the tank. The combination of the light and the nutrients from the flue gases cause the growth of the algae.

The sensors 70 on and around the tank monitor the density of the algae, the light in the tank, the nutrient mix, level of liquidity and other metrics of the system. The sensors 70 transmit this data to the controller which makes the necessary adjustments to the system to ensure optimum algae growth. The scraper 66 can be activated if the light is not being evenly transmitted throughout the tank to scrape the algae from the reflective inner surfaces of the tank.

The algae is then harvested at the appropriate time by draining about half the contents of the tank. Additional liquid and nutrients are added to the remaining algae in the tank to continue the process. The harvested algae is discharged to a processing station where it is dewatered to form a pulp. This pulp can then be further processed to form a biodiesel fuel, to form a solid fuel or for any other suitable use.

This system provides multiple benefits. The flue gases are sequestered from polluting the atmosphere. The flue gases are converted into a useful energy source. The thermal energy from the flue gases are put to beneficial uses by powering the process, by heating the environment around the tanks to the optimum temperature and for processing the harvested algae into a useful fuel. The photoactive process generates oxygen as its byproduct.

These and other embodiments of the present invention are considered to be within the scope of the claimed inventions. The above descriptive embodiment is intended only for explanatory purposes and are not meant to limit the scope of the claimed inventions.

What is claimed is:

1. An algae breeder system wherein said breeder system comprises:
an enclosed tank for containing a liquid medium for growing algae;
a light source for providing light to said tank;
a feedstock inlet on said tank;
a source of flue gases connected to said feedstock inlet for feeding flue gases into the liquid medium in said tank;
a sensor mechanism for measuring the conditions in said tank;
a control mechanism for adjusting the light from said light source, the liquid level in said tank and the flow of the flue gases through said feedstock inlet; and
a controlled environment around said tank.

2. The algae breeder system of claim 1 wherein said controlled environment includes:
an underground enclosure.

3. The algae breeder system of claim 1 wherein said controlled environment includes:
an indoor enclosure.

4. The algae breeder system of claim 1 wherein said light source includes:
a solar collector for providing sunlight to said enclosed tank.

5. The algae breeder system of claim 1 wherein said light source includes:
an artificial light source.

6. The algae breeder system of claim 1 wherein said light source includes:
a combination of a solar collector and an artificial light source to supplement said solar collector.

7. The algae breeder system of claim 1 wherein said system further comprises:
a light modulating system for modulating the light from said light source into said tank.

8. The algae breeder system of claim 1 wherein said system further comprises:
an inner reflective surface in said tank for reflecting light from said light source throughout said tank.

9. The algae breeder system of claim 1 wherein said system further comprises:
a scraping mechanism for cleaning algae from the inner surface of said tank.

10. The algae breeder system of claim 1 wherein said system further comprises:
an environmental control system utilizing thermal energy from the flue gases to control the environment around said tank.

11. The algae breeder system of claim 1 wherein said system further comprises:
a sparging system for sparging said flue gases into small bubbles.

12. A system for converting flue gases into biofuels, wherein said system comprises:
an enclosed tank for containing a liquid medium for growing algae;
a light source for providing light to said tank;
a light modulation system for modulating said light source to optimize the light transmitted in said tank;
a feedstock inlet on said tank;
a source of flue gases connected to said feedstock inlet for feeding flue gases into the liquid medium in said tank;
a sensor mechanism for measuring the conditions in said tank;
a control mechanism for adjusting the light from said light source, the liquid level in said tank and the flow of the flue gases through said feedstock inlet; and
a controlled environment around said tank.

13. The algae breeder system of claim 12 wherein said controlled environment includes:
an underground enclosure.

14. The algae breeder system of claim 12 wherein said controlled environment includes:
an indoor enclosure.

15. The algae breeder system of claim 12 wherein said light source includes:
a solar collector for providing sunlight to said enclosed tank.

16. The algae breeder system of claim 12 wherein said light source includes:
an artificial light source.

17. The algae breeder system of claim 12 wherein said light source includes:
a combination of a solar collector and an artificial light source to supplement said solar collector.

18. The algae breeder system of claim 12 wherein said light source includes:
light strands extending through said enclosed tank.

19. The algae breeder system of claim 18 wherein said system further includes:
grommets that clean algae from said light strands.

20. The algae breeder system of claim 12 wherein said system further comprises:
an inner reflective surface in said tank for reflecting light from said light source throughout said tank.

21. The algae breeder system of claim 12 wherein said system further comprises:
a scraping mechanism for scraping the inner surfaces of said tank.

22. The algae breeder system of claim 1 wherein said system further comprises:
an environmental control system utilizing thermal energy from the flue gases to control the environment around said tank.

23. The algae breeder system of claim 1 wherein said system further comprises:
a sparging system for sparging said flue gases into small bubbles.
